(19) United States
(12) Patent Application Publication
(10) Pub. No.: US 2003/0158255 A1
(43) Pub. Date: Aug. 21, 2003

(54) Use of bismuth subgallate in prevention and/or reduction of skin deterioration

(76) Inventors: Ying-Chieh Tsai, Taipei (TW); Chia-Yen Lin, Taipei (TW); Yen-Ling Shih, Taipei (TW)

Correspondence Address: A. Blair Hughes, McDonnell Boehnen Hulbert & Berghoff, 32nd Floor, 300 S. Wacker Drive, Chicago, IL, 60606, US

(21) Appl. No.: 10/146,016

(22) Filed: May 15, 2002

(51) Int. Cl. A61K 31/29; A61K 7/135

(52) U.S. Cl. 514/503; 424/62

(57) ABSTRACT

The present invention discloses a new use of bismuth subgallate for use in the prevention and/or reduction of skin deterioration. Also disclosed is the synergistic efficacy of bismuth subgallate in combination with borneol in the prevention and/or reduction of skin deterioration.

### BACKGROUND OF THE INVENTION

1. Field of the Invention

The present invention relates to a new use of bismuth subgallate in the prevention and/or reduction of skin deterioration.

2. Description of the Prior Art

Skin is subject to deterioration through dermatological disorders, environmental harm (wind, air conditioning, central heating), or the normal aging process (chronoaging) which may be accelerated by exposure of skin to sun (photoaging). As skin ages, it loses attractive youthful appearance. Aging may be simple chronological passing of the years or photoaging induced by exposure to the sun. Wind, rain or other environmental stresses can cause or aggravate declines due to aging. A variety of skin conditions such as acne, wrinkles, fine lines, pimples, acne lesions, puffiness, psoriasis, age spots and skin discoloration also cause the skin deterioration. Many materials were developed to treat the skin deterioration, such as those disclosed in U.S. Pat. Nos. 6,147,121, 5,910,490, 6,008,454, 6,287,553, 5,561,158 and 5,869,540.

Bismuth subgallate is the product of the reaction between gallic acid, glacial acetic acid and bismuth nitrate and is represented by a molecular formula of C₇H₅BiO₆. It is known as an oral anti-diarrhea agent effective in treating acute or chronic diarrhea by virtue that it can react with H₂S, which is present in large quantities in the intestinal tract due to abnormal fermentation, and thereby alleviate diarrhea and pain caused by gas irritation to the intestinal tract. Bismuth subgallate can also be used as a disinfectant in view of its nature as a benzene derivative.

A pharmaceutical composition for wound healing comprising bismuth subgallate and borneol is disclosed in U.S. Pat. No. 6,232,341. However, none of the prior art teaches or suggests the new use of bismuth subgallate in the prevention and/or reduction of skin deterioration.

### SUMMARY OF THE INVENTION

An object is to provide a method of preventing and/or reducing skin deterioration, comprising applying to skin bismuth subgallate in an effective amount sufficient to prevent and/or reduce the skin deterioration. According to the preferred embodiment of the invention, the method comprising applying to skin bismuth subgallate in combination with borneol in a synergistically effective amount sufficient to prevent and/or reduce the skin deterioration.

Another objective of the invention is to provide a pharmaceutical composition for use in the prevention and/or reduction of skin deterioration, which comprises bismuth subgallate for preventing and/or reducing skin deterioration, in an effective amount sufficient to prevent and/or reduce skin deterioration. According to a preferred embodiment of the invention, the pharmaceutical composition of the invention comprises bismuth subgallate in combination with borneol for synergistically preventing and/or reducing skin deterioration, in a synergistically effective amount sufficient to prevent and/or reduce skin deterioration.

### BRIEF DESCRIPTION OF THE DRAWINGS

FIG. 1 shows the effect of bismuth subgallate (a) and borneol (b) on the proliferation of keratinocytes respectively.

FIG. 2 shows the effect of bismuth subgallate in combination with borneol on the proliferation of fibroblasts.

FIG. 3 shows the inhibition percentage of bismuth subgallate in combination with borneol on tyrosine.

FIG. 4 shows the Lineweaver-Burk's plot of bismuth subgallate in combination with borneol.

### DETAILED DESCRIPTION OF THE INVENTION

It is known that the keratinocyte has fewer intracellular keratohyalin granules and fewer subcellular organelles in older skin compared to younger skin. The fibroblast, by virtue of its activity in the synthesis of extracellular matrix proteins (proteoglycans, collagen fibers and other structural glycoproteins) is the primary constituent in the structural assembly of the dermis. Moreover, it is also known in the art that the tyrosinase inhibitors can be used as a whitening agent for cosmetic products (Shimizu et al., 1998, Planta Medica 64, 408-412; Kojima et al., 1995, Biol. Pharm. Bull. 18(8), 1076-1080). Thus, the proliferation of keratinocyte and fibroblast and the inhibition of tyrosinase may improve the conditions of skin deterioration.

It is surprisingly found in the present invention that bismuth subgallate is useful in the proliferation of keratinocyte and fibroblast and the inhibition of tyrosinase, thereby the skin deterioration can be prevented and/or reduced. In addition, it is found that bismuth subgallate in combination with borneol exhibit a synergistic effect in the prevention and/or reduction of skin deterioration.

Definition

The term “bismuth subgallate” as used herein, refers to the product of the reaction between gallic acid, glacial acetic acid and bismuth nitrate and is represented by a molecular formula of C₇H₅BiO₆.

The term “borneol” as used herein, refers to the product isolated from Dryobalanops aromatica or the like and is represented by the molecular formula C₁₀H₁₇OH.

The term “effective amount” as used herein refers to an amount sufficient to provide an improvement in the prevention and/or reduction of skin deterioration.

The term “skin deterioration” as used herein refers to the conditions of skin aging, photodamage, bumps, pits, wrinkles, fine lines, flaccid texture, acnes, pimples, puffiness, poor color, psoriasis, age spots, skin discoloration, rough skin and dry skin.

Method and Composition for Preventing and/or Reducing Skin Deterioration

One object of the present invention is to provide a method of preventing and/or reducing skin deterioration, comprising applying to skin bismuth subgallate in an effective amount sufficient to prevent and/or reduce the skin deterioration.

According to the invention, the effective amount of bismuth subgallate used in the method of the invention ranges from 0.03 to 40 percent by weight. More preferably, the effective amount of bismuth subgallate ranges from 0.5 to 20 percent by weight. Most preferably, the effective amount of bismuth subgallate ranges from 1.5 to 10 percent by weight.

In one preferred embodiment, the invention provides a method of preventing and/or reducing skin deterioration, comprising applying to skin bismuth subgallate in combination with borneol, in a synergistically effective amount sufficient to prevent and/or reduce the skin deterioration.

According to the invention, the synergistically effective amount of bismuth subgallate in combination with borneol used in the method of the invention range from 0.01 to 30 percent by weight and 0.05 to 10 percent by weight respectively. More preferably, the synergistically effective amount of bismuth subgallate in combination with borneol range from 0.1 to 15 percent by weight, and from 0.1 to 5 percent by weight, respectively. Most preferably, the synergistically effective amount of bismuth subgallate in combination with borneol range from 2 to 6 percent by weight and from 0.5 to 1 percent by weight, respectively.

Another object of the invention is to provide a composition for use in the prevention and/or reduction of skin deterioration, which comprises bismuth subgallate for preventing and/or reducing skin deterioration, in an effective amount sufficient to prevent and/or reduce skin deterioration.

According to the invention, the effective amount of bismuth subgallate used in the composition of the invention ranges from 0.03 to 40 percent by weight. More preferably, the effective amount of bismuth subgallate ranges from 0.5 to 20 percent by weight. Most preferably, the effective amount of bismuth subgallate ranges from 1.5 to 10 percent by weight.

In another preferred embodiment, the invention provides a composition for use in the prevention and/or reduction of skin deterioration, which comprises bismuth subgallate in combination with borneol for use in synergistically preventing and/or reducing skin deterioration, in a synergistically effective amount sufficient to prevent and/or reduce skin deterioration.

According to the invention, the synergistically effective amount of bismuth subgallate in combination with borneol used in the composition of the invention range from 0.01 to 30 percent by weight and 0.05 to 10 percent by weight respectively. More preferably, the synergistically effective amount of bismuth subgallate in combination with borneol range from 0.1 to 15 percent by weight, and from 0.1 to 5 percent by weight, respectively. Most preferably, the synergistically effective amount of bismuth subgallate in combination with borneol range from 2 to 6 percent by weight and from 0.5 to 1 percent by weight, respectively.

Apart from the above-mentioned active ingredients, the compositions according to the present invention may further comprise other traditional agents that are helpful in the prevention and/or reduction of skin deterioration, such as skin aging, photodamage, bumps, pits, wrinkles, fine lines, flaccid texture, acnes, puffiness, poor color, psoriasis, age spots and skin discoloration. For example, the agents selected from the group consisting of alpha-hydroxy acids, antioxidants and tretinoin can be used.

According to the invention, bismuth subgallate or bismuth subgallate in combination with borneol as used in the methods or compositions of the invention can be formulated in the form of emulsion, paste, cream, ointment, and gel paste etc. for topical administration. Suitable carriers used in the formulation of the bismuth subgallate or bismuth subgallate in combination with borneol include, but are not limited to, water, salt solutions, alcohols, polyethylene glycols, gelatin, carbohydrates (such as lactose, amylose or starch), magnesium stearate, talc, silicic acid, viscous paraffin, fatty acid esters, hydroxymethylcellulose, polyvinyl pyrrolidone, etc.

The carrier can be in a wide variety of forms, which can be a diluent, an excipient, a recipient and the like for use in preparing admixtures of a pharmaceutical composition. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g, from about 100 cps to about 200,000 cps and can also be delivered in the form of spray using either mechanical pump containers or pressurized aerosol containers using conventional propellants. These carriers can also be delivered in the form of mousse. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like).

According to the invention, suitable doses of the active ingredients used in the methods and suitable doses of the compositions of the invention may be determined routinely by the medical practitioner or other skilled persons, and include the respective doses discussed in the prior art disclosing bismuth subgallate and borneol that are mentioned hereinbefore, the disclosures in which are hereby incorporated by reference. In any event, a physician, or a skilled person, will be able to determine the actual dosage which will be most suitable for an individual.

Utility

The present invention provides a method and composition which can be used in the prevention and/or reduction of skin deterioration such as skin aging, photodamage, bumps, pits, wrinkles, fine lines, flaccid texture, acnes, pimples, puffiness, poor color, psoriasis, age spots, skin discoloration, rough skin and dry skin. More preferably, the skin deterioration includes acnes, pimples, wrinkles, age spots and skin discoloration. Moreover, the composition as disclosed in the invention can be formulated as pharmaceutical or cosmetic. Given the above, the composition of the invention can condition the rough and dry skin, reduce acnes and pimples, postpone the skin aging, improve skin regeneration and shrink pores.

The following examples further illustrate the present invention, but are not intended to limit the scope of the present invention. The modifications and substitutions known to those skilled in the art are still within the scope and spirit of the present invention.

### EXAMPLES

### Example 1

### Effect of Bismuth Subgallate and Bismuth Subgallate in Combination with Borneol on Proliferation of Keratinocyte

It is known that the keratinocyte has fewer intracellular keratohyalin granules and fewer subcellular organelles in older skin compared to younger skin. Thus, the proliferation of keratinocyte may improve the conditions of skin deterioration.

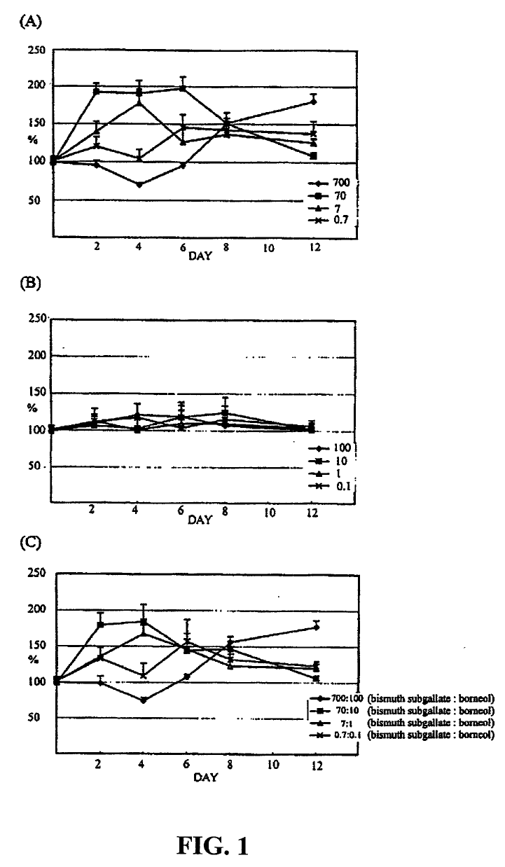

The keratinocyte cells were respectively cultivated with the following groups of agents: (a) 0.7 μg/1 ml, 7 μg/ml, 70 μg/ml and 700 μg/ml of bismuth subgallate; (b) 0.1 μg/ml, 1 μg/ml, 10 μg/ml and 1000 μg/ml of borneol; and (c) 0.7 μg/ml bismuth subgallate in combination with 0.1 μg/ml borneol, 7 μg/ml bismuth subgallate in combination with 1 μg/ml borneol, 70 μg/ml bismuth subgallate in combination with 10 μg/ml borneol and 700 μg/ml bismuth subgallate in combination with 100 μg/ml borneol. After the cells were cultivated after 2, 4, 6, 8, 10 and 12 days respectively, their count was noted to evaluate the proliferation of them. As shown in FIG. 1(a), the cells cultivated with bismuth subgallate exhibits a significant increase in the proliferation of keratinocyte. For example, the cells cultivated with 70 μg/ml bismuth subgallate on the twelfth day had the numbers up to two times of that of control. FIG. 1(b) shows that the borneol has no significant effect on the proliferation of keratinocytes. Moreover, the bismuth subgallate in combination with borneol exhibit synergistic proliferation effect on keratinocyte (see FIG. 1(c).

### Example 2

### Synergistic Effect of Bismuth Subgallate in Combination with Borneol on Proliferation of Fibroblast

Further, the study as to proliferation of fibroblast was carried out to evaluate the effect of the bismuth subgallate in combination with borneol on skin. The fibroblast, by virtue of its activity in the synthesis of extracellular matrix proteins (proteoglycans, collagen fibers and other structural glycoproteins) is the primary constituent in the structural assembly of the dermis. The tested fibroblast cells were obtained from Mackay Memorial Hospital, Taipei, Taiwan, ROC.

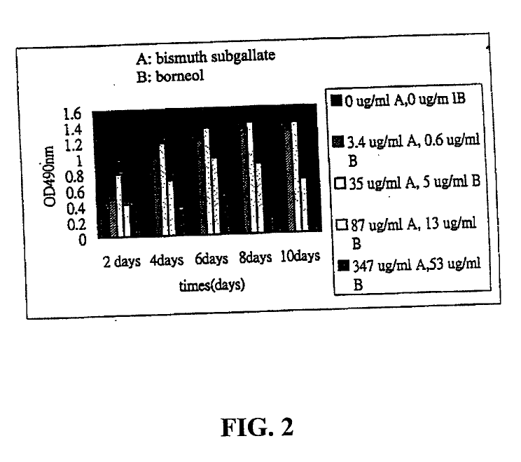

The fibroblast cells were cultivated with 3.4 μg/ml bismuth subgallate and 0.6 μg/ml borneol, 35 μg/ml bismuth subgallate and 5 μg/ml borneol, 87 μg/ml bismuth subgallate and 13 μg/ml borneol, 347 μg/ml bismuth subgallate and 53 μg/ml borneol, respectively. After the cells were cultivated after 2, 4, 6, 8 and 10 days respectively, their count was noted to evaluate the proliferation of them. As shown in FIG. 2, the cells cultivated with low concentration of bismuth subgallate in combination with borneol exhibits significant increase in the proliferation of fibroblast. For example, the cells cultivated with 35 μg/ml bismuth subgallate and 5 μg/ml borneol on the second day have the numbers up to two times of that of control.

### Example 3

### Effect of Bismuth Subgallate in Combination with Borneol on Inhibition of Tyrosinase

The study as to inhibition of tyrosine was carried out to evaluate the effect of the bismuth subgallate in combination with borneol on skin. Melanin biosynthesis occurs in melanocytes and involves the oxidation and polymerization of tyrosine to 3,4-dihydroxyphenylalanine (dopa) and dopa to melanin. Thus, the tyrosine inhibitors can be used as the whitening agents for cosmetic products through the block of the production of melanin.

0.09% of L-DOPA in 0.1M sodium phosphate buffer, pH 6.8 with or without 120 μM, 240 μM, 400 μM, 600 μM, 800 μM and 1600 μM of bismuth subgallate in combination with borneol and 50 μl, 135U/ml tyrosinease were used to test the inhibition on tyrosine. The resulting solutions were reacted at 37° C. for 15 minutes and then detected by spectrophotometer at 475 nm. The inhibition percentage of the bismuth subgallate in combination with borneol on tyrosinase was calculated by the following formula:

Inhibition percentage (%)=[(A−B)−(C−D)]/(A−B)×100

A=OD₄₇₅ of the solution without bismuth subgallate in combination with borneol

B=OD₄₇₅ of the solution without tyrosine and bismuth subgallate in combination with borneol

C=OD₄₇₅ of the solution with bismuth subgallate in combination with borneol

D=OD₄₇₅ of the solution with bismuth subgallate in combination with borneol but without tyrosine

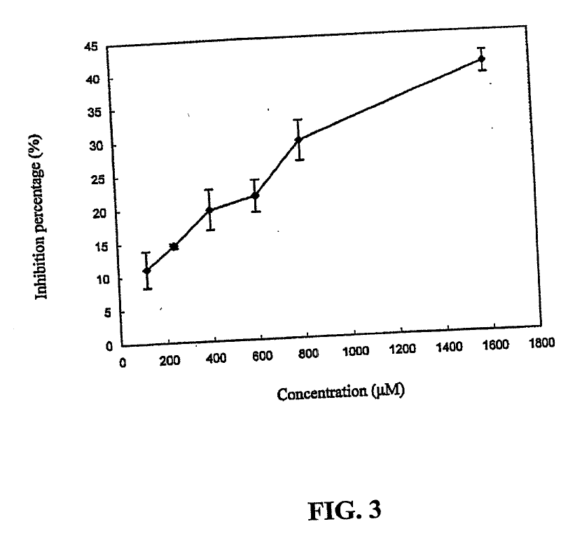

The inhibition percentages of 120 μM, 240 μM, 400 μM, 600 μM, 800 μM and 1600 μM of bismuth subgallate in combination with borneol were 11.1%, 14.6%, 19.7%, 21.5%, 29.5% and 40.3% and the IC₅₀ value was more than 1600 μM (see FIG. 3).

Further, the enzyme kinetic analysis shows that the bismuth subgallate in combination with borneol is a competitive inhibitor of the tyrosinase.

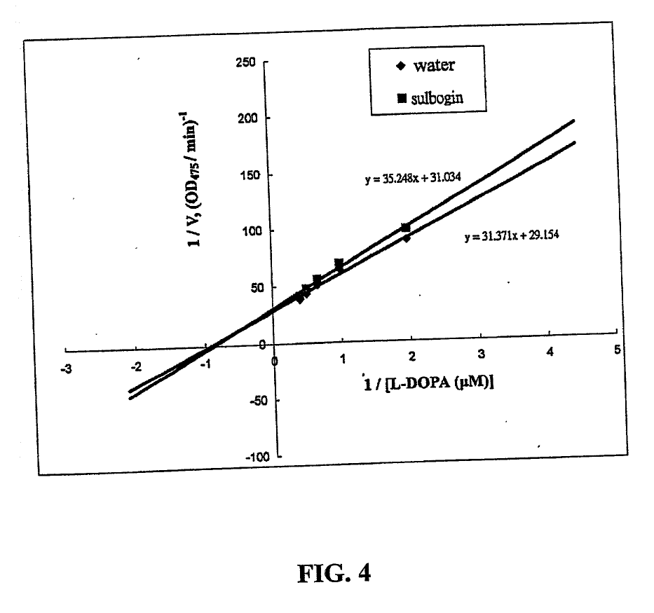

50 μl, various concentration of L-DOPA in 0.1M sodium phosphate buffer and 50 μl, 120 μM of the bismuth subgallate in combination with borneol were mixed and then added in 96 well plate. 50 μl, 135U/ml of tyrosinease was added to each concentration of L-DOPA solution. The resulting solutions were added to the plate and reacted at 37° C. for 15 minutes. The Lineweaver-Burk's plot of the bismuth subgallate in combination with borneol in the inhibition of tyrosinase was shown in FIG. 4. The Ki value of the bismuth subgallate in combination with borneol is 971 μM.

### Example 4

### Application of Bismuth Subgallate in Combination with Borneol on Skin

Five persons A, B, C, D and E applied the composition comprising bismuth subgallate in combination with borneol on their faces. After applying the bismuth subgallate in combination with borneol for two months, the person A had a reduction on pimples and acnes and her skin color became whitened; the person B had an improvement on skin color; the skin fineness of the person C was better; the dry condition of the skin of the person D was improved; and the person E has an improvement on skin allergy.

What is claimed is:

1. A method of preventing and/or reducing skin deterioration, comprising applying to skin bismuth subgallate in an effective amount sufficient to prevent or reduce the skin deterioration.

2. The method according to claim 1, wherein the amount of bismuth subgallate ranges from 0.03 to 40 percent by weight.

3. The method according to claim 1, wherein the amount of bismuth subgallate ranges from 0.05 to 20 percent by weight.

4. The method according to claim 1, wherein the amount of bismuth subgallate ranges from 1.5 to 10 percent by weight.

5. The method according to claim 1, wherein said skin deterioration is the condition selected from the group consisting of aging, photodamage, bumps, pits, wrinkles, fine lines, flaccid texture, acnes, pimples, puffiness, poor color, psoriasis, age spots, skin discoloration, rough skin and dry skin.

6. The method according to claim 5, wherein said conditions are selected from the group consisting of acnes, pimples, wrinkles, age spots and skin discoloration.

7. A method of preventing or reducing skin deterioration, comprising applying to skin bismuth subgallate in combination with borneol, in a synergistically effective amount sufficient to prevent and/or reduce the skin deterioration.

8. The method according to claim 7, wherein the amount of bismuth subgallate and borneol range from 0.01 to 30 percent by weight and from 0.05 to 10 percent by weight, respectively.

9. The method according to claim 7, wherein the amount of bismuth subgallate and borneol range from 0.1 to 15 percent by weight and from 0.1 to 5 percent by weight, respectively.

10. The method according to claim 7, wherein the amount of bismuth subgallate and borneol range from 2 to 6 percent by weight and from 0.5 to 1 percent by weight, respectively.

11. The method according to claim 7, wherein said skin deterioration is the conditions selected from the group consisting of aging, photodamage, bumps, pits, wrinkles, fine lines, flaccid texture, acnes, pimples, puffiness, poor color, psoriasis, age spots and skin discoloration.

12. The method according to claim 7, wherein said conditions are selected from the group consisting of acnes, pimples, wrinkles, age spots and skin discoloration.

13. A composition for use in the prevention or reduction of skin deterioration, which comprises bismuth subgallate for preventing and/or reducing skin deterioration, in an effective amount sufficient to prevent and/or reduce skin deterioration.

14. The composition according to claim 13, wherein the amount of bismuth subgallate ranges from 0.03 to 40 percent by weight.

15. The composition according to claim 13, wherein the amount of bismuth subgallate ranges from 0.5 to 20 percent by weight.

16. The composition according to claim 13, wherein the amount of bismuth subgallate ranges from 1.5 to 10 percent by weight.

17. The composition according to claim 13, which can be used as pharmaceutical or cosmetic.

18. The composition according to claim 13, wherein said skin deterioration is the condition selected from the group consisting of aging, photodamage, bumps, pits, wrinkles, fine lines, flaccid texture, acnes, pimples, puffiness, poor color, psoriasis, age spots and skin discoloration.

19. The composition according to claim 13, wherein said conditions are selected from the group consisting of acnes, pimples, wrinkles, age spots and skin discoloration.

20. A composition for use in preventing and/or reducing sin deterioration which comprises bismuth subgallate in combination with borneol for synergistically preventing and/or reducing skin deterioration, in a synergistically effective amount sufficient to prevent and/or reduce skin deterioration.

21. The composition of claim 20, wherein the amount of bismuth subgallate and borneol range from 0.01 to 30 percent by weight and from 0.05 to 10 percent by weight, respectively.

22. The composition of claim 20, wherein the amount of bismuth subgallate and borneol range from 0.1 to 15 percent by weight and from 0.1 to 5 percent by weight, respectively.

23. The composition of claim 20, wherein the amount of bismuth subgallate and borneol range from 2 to 6 percent by weight and from 0.5 to 1 percent by weight, respectively.

24. The composition according to claim 20, which can be used as pharmaceutical or cosmetic.

25. The composition according to claim 20, wherein said skin deterioration is the condition selected from the group consisting of aging, photodamage, bumps, pits, wrinkles, fine lines, flaccid texture, acnes, pimples, puffiness, poor color, psoriasis, age spots and skin discoloration.

26. The composition according to claim 20, wherein said skin deterioration is the condition selected from the group consisting of acnes, pimples, wrinkles, age spots and skin discoloration.
